(19) United States
(12) Patent Application Publication
(10) Pub. No.: US 2024/0279690 A1
(43) Pub. Date: Aug. 22, 2024

(54) PROCESS FOR FRACTIONATION OF PHOTOSYNTHETIC BIOMASS AND RELATED SYSTEMS AND METHODS

(71) Applicant: Filament Biosciences, LLC., Cascade, IA (US)

(72) Inventor: Marcella Roe, Cascade, IA (US)

(21) Appl. No.: 18/444,307

(22) Filed: Feb. 16, 2024

Related U.S. Application Data
(60) Provisional application No. 63/446,317, filed on Feb. 16, 2023.

(51) Int. Cl. C12P 7/16; C12P 7/04; C12P 17/06; C12P 17/10

(52) U.S. Cl. CPC C12P 7/16; C12P 7/04; C12P 17/06; C12P 17/10

(57) ABSTRACT

A chemical extraction method comprising exposing a biomass to a protein-denaturing solution to produce a suspended biomass mixture, separating the solubilized biomass mixture into a supernatant phase and a decolorized biomass phase, hydrolyzing the decolorized biomass phase into a hydrolyzed product, fermenting the hydrolyzed product into an alcoholic solvent, mixing the alcoholic solvent and supernatant phase to produce a product suspension, and fractioning the product suspension into a bottom phase, middle phase, and top phase.

### CROSS-REFERENCE TO RELATED APPLICATION(S)

This application claims the benefit under 35 U.S.C. § 119(e) to U.S. Provisional Application No. 63/446,317, filed Feb. 16, 2023, and entitled Process for Fractionation of Chlorophyll and/or Carotenoid-Containing Biomass, which is hereby incorporated herein by reference in its entirety for all purposes.

### TECHNICAL FIELD

The disclosure relates to processing algal, cyanobacterial, and plant biomass into various constituent components that may be used in further processes.

### BACKGROUND

Chlorophyll is any of several related natural green pigments that include chlorophyll A, B, C1, C2, D, and F found in photosystems of cyanobacteria, algae, and plants. Chlorophylls absorb light most strongly in the blue wavelength portion of the electromagnetic spectrum as well as the red wavelength portion. Conversely, chlorophyll is a poor absorber of green and near-green portions of the spectrum. Chlorophyll consists of a tetrapyrrole ring system that include a central magnesium atom, porphyrin ring, and a long phytol chain. Chlorophyll is an essential and valuable compound in many products.

When extracted intact under controlled and non-degrading conditions, chlorophyll can be used as an additive in pharmaceutical and cosmetic products, as a natural green food coloring agent, and as an antioxidant, antibacterial, anti-inflammatory, antiviral, and antimutagenic nutraceutical food additive. Chlorophyll is often transformed to sodium copper chlorophyll, which is widely used in the area of food additives, colorants, etc. Chlorophyll as a food ingredient is gaining attention due to increasing consumer demand for natural and sustainable foods and clean-label market trends. Furthermore, chlorophyll that has been extracted intact can be enzymatically cleaved or partitioned under mild ambient temperature into various health-promoting by-products, including pheophorbide A which has been demonstrated to possess anti-tumor properties.

In contrast, chlorophyll is also non-essential nuisance compound that prevents the reproducibility and accurate measurement of certain in vitro bioassays due to its UV/Vis absorbance, fluorescence properties, and tendency to precipitate in aqueous media. These characteristics further warrant selective removal of chlorophyll from biomass.

Chlorophyll is sensitive to heat, light, acids, and alkali. Chlorophyll, especially when out of cellular chloroplasts and exposed, extracted, purified, and dried via organic solvents, is sensitive to pH variations, heat, oxygen, and light, causing the chlorophyll and derived compounds to degrade easily by various mechanisms into non-nutraceutical, unpalatable, discolored, and even toxic by-products.

Carotenoids are red, yellow, and orange lipophilic tetraterpenoid natural pigments universally synthesized as secondary metabolites by terrestrial and aquatic photoautotrophs, including plants (fruits, vegetables), fungi, bacteria, microalgae, and macroalgae. In microalgae and plant cells, carotenoids are biosynthesized and stored in plastids, where they play essential roles in oxygenic photosynthesis (light harvesting), photoprotection (detoxification of free radicals generated during photosynthesis) and signaling pathways. In plants, carotenoids serve as a precursor to the biosynthesis of phytohormones such as abscisic acid and strigol-acetones, which play a crucial role in regulating several plant developmental and adaptation processes. Plastids are crucial in controlling carotenogenic activity, pigment diversity, and carotenoids stability. Cultivational, environmental (light intensity, drought, salinity, and chilling stresses), and genetic factors significantly influence the carotenoid contents of plants.

Carotenoids have important implications for human health and the food industry due to their antioxidant and functional properties. In addition to central functions in plants and other photosynthetic microbes, carotenoids play an essential role by providing a dietary source of provitamin A (e.g., α- and β-carotene and β-cryptoxanthin). The antioxidant activities of carotenoids regulate oxidative stress (stabilize cellular membranes) and inflammatory mediators, thus protecting against metabolic syndromes (MetS: CVD and T2D), cancer, neurodegenerative diseases, and photooxidative damage to the skin and eyes. As most animals are incapable of biosynthesizing carotenoids, animals need to obtain carotenoids from their diets.

Carotenoids tend to oxidize, which limits their ability to withstand exposure to heat, light, acids, and long extraction times. Carotenoids are stable in biological systems because they form protein complexes in their natural environment and because of the presence of natural antioxidants in biological systems. In isolated or pure form, carotenoids in the presence of oxygen and light degrade rapidly. The ability of carotenoids to interact with free radicals, including peroxyl radicals, results from systems of conjugated double bonds in carotenoids, which render the carotenoids open to oxidation.

In addition to chlorophylls and carotenoids, photosynthetic biomass includes soluble proteins and membrane-bound water-insoluble proteins that have wide ranging applications, including cosmetics and food ingredient applications. Photosynthetic biomass also includes polysaccharides, typically in the cell wall, such as cellulose, with myriad applications in the food, feed, textile, cosmetics, biomedical, and electronics industries. Photosynthetic biomass also includes lipids that include but are not limited to neutral lipids as pre-cursors to biofuels and valuable long-chain polyunsaturated omega-3 free fatty acids like DHA or EPA found in the microalgae Porphyridium Purpurum and Nannochloropsis salina, for instance. These lipids found in photosynthetic biomass have food and animal feed product applications.

With all things considered, there is a need for low-cost, fast, efficient, environmentally benign, non-toxic processes for the simultaneous purification of intact chlorophyll, carotenoids, proteins, polysaccharides, lipids/fatty-acids, and other valuable components from photosynthetic biomass.

### BRIEF SUMMARY

Disclosed herein are various methods and related systems and devices for fractionation of biomass containing proteins, polysaccharides, carotenoids, chlorophyll, and/or lipids.

In Example 1, a chemical extraction method comprising exposing a biomass to a protein-denaturing solution to produce a suspended biomass mixture, separating the solubilized biomass mixture into a supernatant phase and a decolorized biomass phase, hydrolyzing the decolorized biomass phase into a hydrolyzed product, fermenting the hydrolyzed product into an alcoholic solvent, mixing the alcoholic solvent and supernatant phase to produce a product suspension, and fractioning the product suspension into a bottom phase, middle phase, and top phase.

Example 2 relates to the chemical extraction method of any of Examples 1 and 3-11, wherein separating the solubilized biomass mixture is accomplished with gravimetric settling.

Example 3 relates to the chemical extraction method of any of Examples 1-2 and 4-11, wherein separating the solubilized biomass mixture is accomplished with centrifugation.

Example 4 relates to the chemical extraction method of any of Examples 1-3 and 5-11, further comprising washing the solubilized biomass mixture and re-exposing the solubilized biomass mixture to a protein-denaturing solution.

Example 5 relates to the chemical extraction method of any of Examples 1-4 and 6-11, wherein the alcoholic solvent is butanol.

Example 6 relates to the chemical extraction method of any of Examples 1-5 and 7-11, wherein fractioning the product suspension is accomplished by gravimetric settling.

Example 7 relates to the chemical extraction method of any of Examples 1-6 and 8-11, further comprising filtering the bottom layer into proteins and permeate, transesterifying the middle phase with fatty esters, and drying the top layer into a powder.

Example 8 relates to the chemical extraction method of any of Examples 1-7 and 9-11, wherein the powder is substantially chlorophyll.

Example 9 relates to the chemical extraction method of any of Examples 1-8 and 10-11, further comprising enzymatically processing the powder into a tocopherol, pheophorbide B, or phytol.

Example 10 relates to the chemical extraction method of any of Examples 1-9 and 11, wherein the protein-denaturing solution is a PUTTS buffer.

Example 11 relates to the chemical extraction method of any of Examples 1-10, wherein the biomass is one or more of macroalgae, microalgae, cyanobacteria, and terrestrial plants.

In Example 12, a biomass separation system comprising an agitation device configured to contact a biomass and a PUTTS solution to produce a solubilized biomass mixture, a first separation device capable of separating the solubilized biomass mixture based on density into a supernatant and decolorized biomass, a hydrolysis reactor configured to hydrolyze the decolorized biomass, a fermenter configured to ferment the decolorized biomass into an alcoholic solution, a mixing vessel configured mix the alcoholic solution and supernatant into a product suspension, and a second separation device for separating the product suspension into a bottom phase, middle phase, and top phase.

Example 13 relates to the biomass separation system of any of Examples 12 and 14-17, wherein the first separation device is a centrifuge or a settling tank.

Example 14 relates to the biomass separation system of any of Examples 12-13 and 15-17, further comprising a filter configured to filter the bottom layer into proteins and permeate.

Example 15 relates to the biomass separation system of any of Examples 12-14 and 16-17, further comprising a dryer configured to separate the top layer into alcohol solvent and a powder.

Example 16 relates to the biomass separation system of any of Examples 12-15 and 17, wherein the powder is substantially chlorophyll.

Example 17 relates to the biomass separation system of any of Examples 12-16, further comprising an enzymatic reactor configured to enzymatically react the chlorophyll into tocopherols, pheophorbide B, or phytol.

In Example 18 a method for fractioning photosynthetic biomass, comprising solubilizing photosynthetic biomass in a first solvent, separating the solubilized photosynthetic biomass into a supernatant and decolorized biomass, hydrolyzing the decolorized biomass, fermenting the hydrolyzed decolorized biomass, missing the second solvent with the supernatant, fractioning the supernatant mixed with the second solvent into top, middle, and bottom layers, and extracting chlorophyll, catenoids, lipids and fatty acids, proteins, polysaccharides from the top, middle, and bottom layers, wherein the first solvent is selected from a relatively hydrophilic aqueous surfactant- and protein denaturant-based solvent, a relatively hydrophilic aqueous ionic liquid-based solvent, a relatively hydrophilic aqueous surfactant- and/or protein denaturant-based deep eutectic solvent, and an immiscible, relatively hydrophobic deep eutectic solvent, wherein the second solvent is selected from an immiscible, relatively hydrophobic polar alcohol; an immiscible, relatively hydrophobic polymer-based solvent; an immiscible, relatively hydrophobic aqueous ionic liquid-based solvent; an immiscible, relatively hydrophobic, deep eutectic solvent; and an immiscible, relatively hydrophobic aqueous ionic liquid-based solvent.

Example 19 relates to the method of any of Examples 18 and 20, wherein the first solvent is PUTTS buffer and the second solvent is n-butanol.

Example 20 relates to the method of any of Examples 18-19, wherein deep eutectic solvents are used to de-water and recover polar alcohol solvents.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a process diagram showing the fractionation of photosynthetic biomass, according to one implementation.

FIG. 2 shows biomass after extraction using PUTTS buffer and Butanol (A) (left), Methanol (B), and Hexane (C) at a 1:1 v:v ratio.

FIG. 3 shows PUTTS:Butanol (1:1) extraction after centrifugation of biomass from nitrogen-replete Stramenopile algae (A), nitrogen deplete Stramenopile algae (B), nitrogen deplete Muriella zofingeinsis (C), and nitrogen deplete Haemalococcus pluvialis (D).

FIG. 4A shows triplicate PUTTS:Butanol (1:1 v:v) extraction of residual raffinate resulting from extraction of water-soluble protein (including phycocyanin) from the dry biomass of Arthrospria platensis that was PEF-treated, solubilized in PUTTS, centrifuged, and separated as supernatant from a polysaccharide-rich pellet.

FIG. 4B shows separated fractions of Butanol (three leftmost tubes), PUTTS layer also including a washed and hydrolyzed polysaccharide layer (three rightmost tubes), and dried central lipid interphase (tubes in front).

FIG. 4C shows the PUTTS layer after passing through 3000 kD MWCO centrifuge filter.

FIG. 5 shows extractions of micro fluidized, N-replete (low-oil) Tribonema viride.

FIG. 6 shows extractions of micro fluidized, N-deplete (high-oil), Tribonema viride.

FIG. 7 shows extractions of undisrupted, N-replete (low-oil) Tribonema viride.

FIG. 8 shows extractions of undisrupted, N-deplete (high-oil), Tribonema viride.

FIG. 9 shows extractions of micro fluidized, N-replete Hematococcus pluvialis.

FIG. 10 shows extractions of micro fluidized, N-replete Chlorella vulgaris.

FIG. 11 shows extractions of micro fluidized, N-replete Porphyridium cruentum.

FIG. 12 shows extractions of micro fluidized, N-replete (low-oil) Tribonema viride.

FIG. 13 shows extractions of micro fluidized, N-deplete (high-oil), Tribonema viride.

FIG. 14 shows extractions of undisrupted, N-replete (low-oil) Tribonema viride.

FIG. 15 shows extractions of undisrupted, N-deplete (high-oil), Tribonema viride.

FIG. 16 shows extractions of micro fluidized, N-replete Hematococcus pluvialis.

FIG. 17 shows extractions of micro fluidized, N-replete Chlorella vulgaris.

FIG. 18 shows extractions of micro fluidized, N-replete Porphyridium cruentum.

FIG. 19 shows extractions of micro fluidized, N-replete Porphyridium cruentum.

### DETAILED DESCRIPTION

Discussed herein are various methods and related systems and devices for fractioning and decolorizing biomass. The various implementations are also configured to purify proteins, polysaccharides, chlorophylls, carotenoids, and/or lipids from biomass via inexpensive, recoverable, reusable, non-toxic, environmentally benign solvents. In various implementations, the methods and systems enable high-yielding fractionation of biomass derived from photosynthetic organisms, including but not limited to plant, microalgae, macroalgae, and cyanobacteria, into separate high-purity components via low-cost, low-temperature, low-energy extraction methods that use low-cost, low-viscosity, recoverable, reusable, non-toxic, and environmentally benign solvents.

Additionally, disclosed herein is a solvent mixture that is low-cost, recoverable, reusable, non-toxic, environmentally benign, and effective for efficient, selective, and rapid extraction, separation, and purification of chlorophyll, water-soluble proteins, water-insoluble proteins, polysaccharides, carotenoids, and lipids from photosynthetic biomass.

In various implementations, photosynthetic biomass is processed into separate fractions that include but are not limited to membrane-bound water-insoluble proteins, lipids (i.e. neutral, polar, and/or associated fatty acids), carotenoids, chlorophylls, and polysaccharides. In certain implementations, subsequent to separation, various of the individual fractions undergo additional processing for separation, purification, and/or conversion of individual fraction into end use components.

In various implementations, the polysaccharides, from the biomass, may be hydrolyzed to sugars that can be fermented to produce alcohol-based solvents, optionally used in the de-colorizing and purifying extraction-based processes. Purified cellulose polysaccharide can be further processed to nanocellulose with broad industrial, biomedical and energy storage applications. Purified protein can be used directly or hydrolyzed for a myriad of applications including food, feed, beverage, cosmetics, textiles, biomaterials, etc. Purified fatty acids can be used as precursors to biofuels and biomaterials as well as in food and beverage applications. Purified carotenoids have nutraceutical food applications. Purified chlorophyll and its degradation by-products, such as pheophorbide A, have uses as natural green colorant and nutraceutical ingredients. Various additional uses of the purified components of the biomass are possible and would be known and appreciated by those of skill in the art.

The various methods and processes described include the application and use of efficient, selective, industrially scalable, cost-effective three-phase partitioning extractions for the simultaneous, cost-effective, removal of chlorophyll, carotenoids, polysaccharides, protein, and lipid/fatty acids, and other components from photosynthetic biomass. These extractions include the use of various non-toxic, environmentally benign, recoverable, two-solvent combinations. Examples for top-layer solvents may include one or more of aqueous or non-aqueous formulations involving alcohols (e.g. n-butanol, tert-butanol, and others), hydrophobic ionic liquid solvents (e.g. 1-butyl-3-methyl-imidazolium, 1-ethyl-3-methylimidazolium dihydrogen phosphate), hydrophobic deep eutectic solvents (e.g., glycerol: choline chloride glycerol/methyltrioctylammonium chloride, 1-butanol:[TOPO], acetic acid:dl-menthol, lactic acid:serine, choline chloride:Ph-EtOH, or [HFIP]:Triton X-100), and polymers (e.g. polyethylene glycol 1000 (PEG1000), polypropylene glycol 5000, and others). Examples of bottom-layer solvents may include one or more of aqueous formulations involving surfactant/urea-containing solvent (e.g. PUTTS buffer, methyltrioctylammonium chloride, and others), hydrophilic ionic liquids (e.g. choline acetate, choline dihydrogen phosphate, choline chloride, ethyl methyl imidazolium dibutyl phosphate and others), and hydrophilic deep eutectic solvents (e.g. choline chloride-urea, choline acetate-urea, and others).

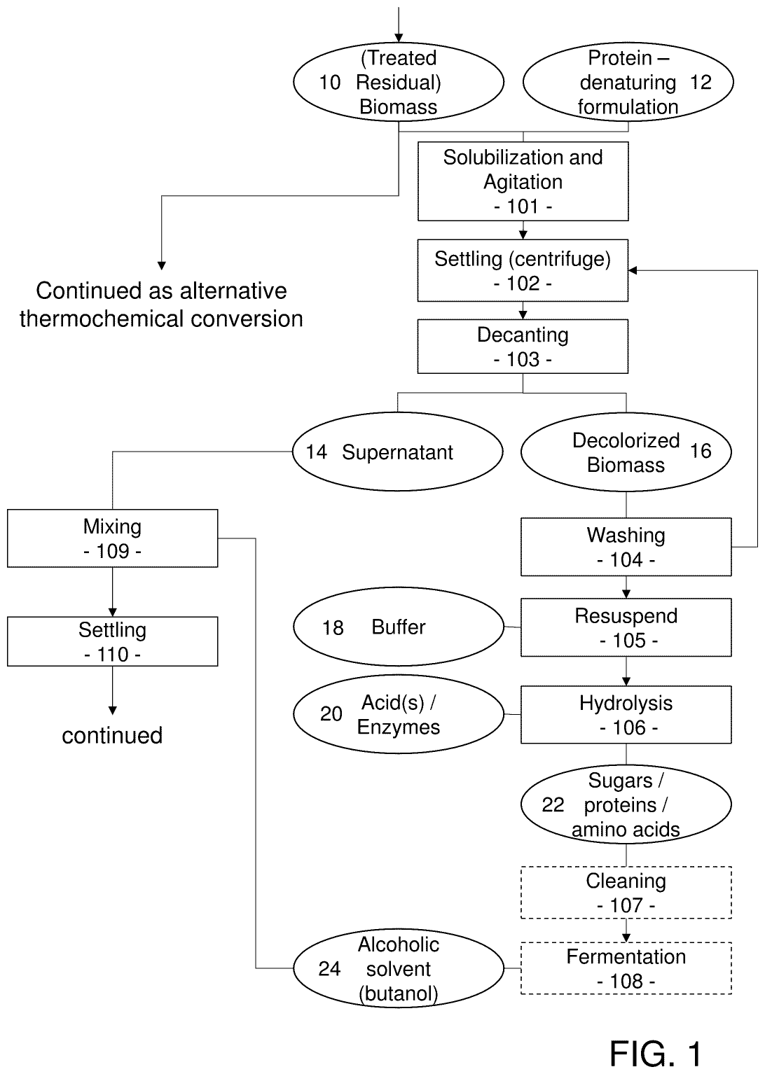

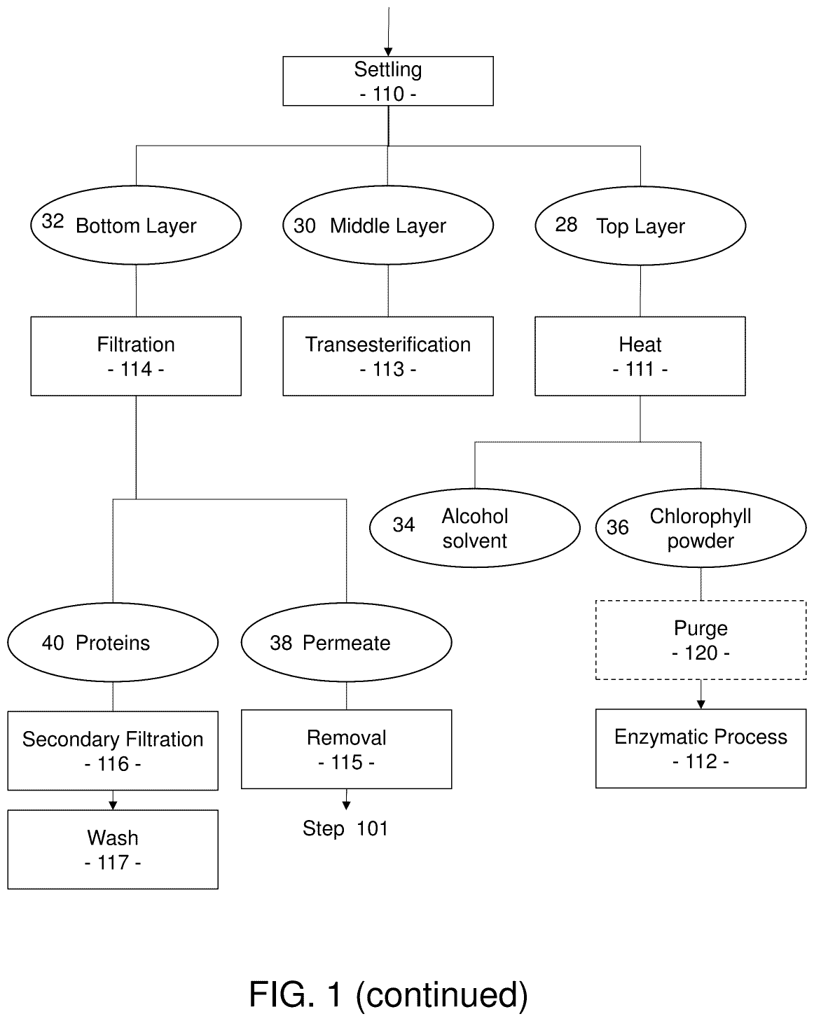

The processes and methods described herein include various steps and sub steps each of which is optional and may be performed in any order or not at all. Various steps may be performed iteratively, simultaneously with other steps or sub steps, or in sequence with other steps or sub steps. An outline of the process steps, including optional steps, is shown in FIG. 1.

In a first optional step, biomass 10 is solubilized (box 101) by addition of either a protein denaturing formulation 12 or a protein non-denaturing formulation 12. In this step, the combined biomass 10 and protein denaturing formulation 12 are agitated to aid in solubilization (box 101). The solubilization and agitation step (box 101) may include vortexing, inversion, or the like. In certain implementations, the agitation may be for about 20 seconds, or up to 2 minutes. In some implementations, agitation may be up to about 10 minutes.

The ratio of denaturing formulation 12 volume to biomass 10 can include but is not limited to about 20:1 (50 mL to 2.5 mL). Various different ratios are possible and would be recognized by those of skill in the art.

In various implementations, a hydrophilic protein denaturing solution 12 includes but is not limited to (a) a PUTTS Buffer formulation comprised of 8M urea, 1% (v/v) Triton-X, 0.2% (w/v) Sarkosyl, 100 mM NaH2PO4, 10 mM Tris-HCl buffer [pH 7.5], (b) an ionic liquid such as Choline Acetate or Choline Dihydrogen Phosphate (c) an aqueous solution of a deep eutectic solvent, that includes, but is not limited to, Choline Chloride-Urea (2:1 molar ratio), Choline Acetate-Urea (1:2 molar ratio), and Choline Dihydrogen Phosphate-Urea (1:2 molar ratio). Various alternative ratios and combinations are possible and would be appreciated.

Various alternative protein denaturing solutions 12 containing surfactants surfactants including but not limited to methyltrioctylammonium chloride, sarkosyl, Triton X100, other anionic, cationic, and/or nonionic detergents, aqueous urea, guanidine-hydrochloride solution, sodium dodecyl sulfate (SDS) solution and buffers such as TrisHCL and others are known and recognized by those of skill in the art.

In various implementations, a hydrophilic protein non-denaturing solution 12 includes but is not limited to a hydrophilic solution comprised of aqueous ionic liquid solvent, such as Choline Chloride, Choline Acetate, and Choline Dihydrogen Phosphate. Various alternative protein denaturing solutions 12 are known and recognized by those of skill in the art.

In certain implementations, the biomass 10 can be derived from many organisms including but not limited to macroalgae, microalgae, cyanobacteria, and/or terrestrial plants. The biomass 10 can be in any number of states including, among others, wet paste or dry. The biomass 10 can be untreated or treated previously as raffinate via cell-lytic methods that include but are not limited to high-pressure homogenization, mechanical shear or bead-beating, micro-fluidization, freeze-thaw cycling, ultrasonication, microwave, ozonation, and/or cell permeabilization methods that include but are not limited to static or continuous pulsed electric field. Various alternative treatments of the biomass or state of the biomass may be known and recognized by those of skill in the art.

In another optional step, the solubilized biomass mixture is allowed to gravimetrically settle (box 102) and/or is centrifuged (box 102) to separate the mixture into a supernatant 14 and decolorized biomass pellet 16. The centrifugation may be at a sufficient force, duration, and temperature for such separation/settling to occur. In one exemplary implementation, the centrifuge operates at 4500×g, for ten minutes, at 4° C. Various alternative settings and conditions (e.g. temperatures, concentrations, etc.) are possible and would be appreciated by those of skill in the art. As would be understood, the decolorized biomass pellet 16 could take many forms, resulting from different styles of separation. Examples, as would be known in the art, include a dense, dry pellet from a benchtop centrifuge, a wet slurry produced from the heavy phase of a decanter centrifuge or high-speed disc stack centrifuge, or a solid mass of varying dryness from the solids discharge of a dewatering centrifuge.

In a further optional step, the supernatant 14 is decanted (box 103), aspirated away from or other otherwise separated from a settled, visually decolorized, solid pellet of biomass 16.

The pellet 16 may then be optionally washed (box 104). The washing (box 104) may optionally be done with deionized water or reverse osmosis water. The washing (box 104) may also include mixing the pellet 16 with water and inversion and re-centrifuged (box 102). The washing step (box 104) may be performed one time or iteratively, as would be understood. The washing step (box 104) can also be performed continuously based on residence time of portions of the pellet 16 in flowing through a system such as a continuous filter and dryer.

In another optional step, the pellet 16 is re-suspended (box 105) in a buffer 18. The buffer 18 may be aqueous 20 mM Sodium Phosphate Buffer, pH 7.0 or other buffer that is compatible for enzymatic hydrolysis and/or acid-hydrolysis of the pellet. Various alternative buffers would be known and appreciated by those skilled in the art. The ratio of re-suspension buffer 18 volume to pellet mass 16 may be about 2:1 or alternative quantities sufficient to resuspend the pellet.

In a further optional step, various acids and/or enzyme(s) 20 are added to the pellet 16 to hydrolyze (box 106) various components of the pellet 16. For example, polysaccharides may be hydrolyzed (box 106) to monomeric sugars or other sugars, proteins may be hydrolyzed (box 106) to amino acids, and/or other materials in the pellet 16 may also be hydrolyzed (box 106) into component parts, as would be understood. Hydrolysis (box 106) may have various reaction conditions and durations, as would be appreciated. For example, hydrolysis (box 106) may occur at 37° C., with gentle rocking, for 6 hours before neutralization. Alternatively, hydrolysis (box 106) may occur at 80° C. for 1 hour.

Enzymes 20 may include, but are not limited to, lysozyme, pectinase, glucanase, alcalase, papain, chitninase, chitosanase, cellulases, snailase, sulfatase, and others added in sufficient units, as would be understood. Acids 20 may include 72% H2SO4, 2M or 6M TFA, and/or 6M HCl. Various further acids 20 are possible and would be recognized by those of skill in the art.

In a further optional step, the hydrolytic reaction products 22 (sugars/proteins/amino acids 22) may optionally be cleaned (box 107). Methods for cleaning (box 107) may include use of activated carbon adsorption to remove enzymatic inhibitor content and/or other impurities. Various additional or alternative cleaning steps or methods may be implemented, as would be appreciated by those skilled in the art. Alternatively the hydrolytic reaction products 22 may be left unwashed.

A further optional step includes fermenting (box 108) the washed or unwashed hydrolytic reaction products 22. Fermentation (box 108) may occur at various conditions and durations, as would be understood. Fermentation (box 108) may be done by various microbial organisms that convert hydrolyzed material 22 to solvents 24 that include but are not limited to butanol. Exemplary organisms for fermentation (box 108) may include Escherichia coli, Synechococcus elongatus PCC 7942 and Clostridium acetobutylicum, while others are possible and would be understood.

In various implementations, the hydrophobic solvent 24 may be, but is not limited to, (a) one or more alcohols that include, but are not limited to n-butanol 24 obtained by fermentations (box 108) or other alcohol, (b) an aqueous polymer solution that includes, but is not limited to polyethylene glycol 1000 (PEG1000), (c) an aqueous solution of ionic liquid that includes, but is not limited to 1-ethyl-3-methylimidazolium dihydrogen phosphate, (d) an aqueous solution of deep eutectic solvents that includes, but is not limited to glycerol: Choline Chloride, glycerol:methyltrioctylammonium chloride, 1-butanol:[TOPO], acetic acid:dl-menthol, lactic acid:serine, Choline Chloride:Ph-EtOH, or [HFIP]:Triton X-100. The hydrophobic solvent 24 is immiscible in a solubilization formulation like PUTTS Buffer that is mixed (box 109) with the supernatant 14 obtained from the settling and decanting steps (boxes 102 and 103) discussed above. In various implementations the supernatant 14 and hydrophobic solvent 24 are mixed (box 109) and optionally agitated. In various implementations, the supernatant 14 and hydrophobic solvent 24 may be mixed (box 109) at a ratio of about 1:1, while other ratios are possible and would be understood.

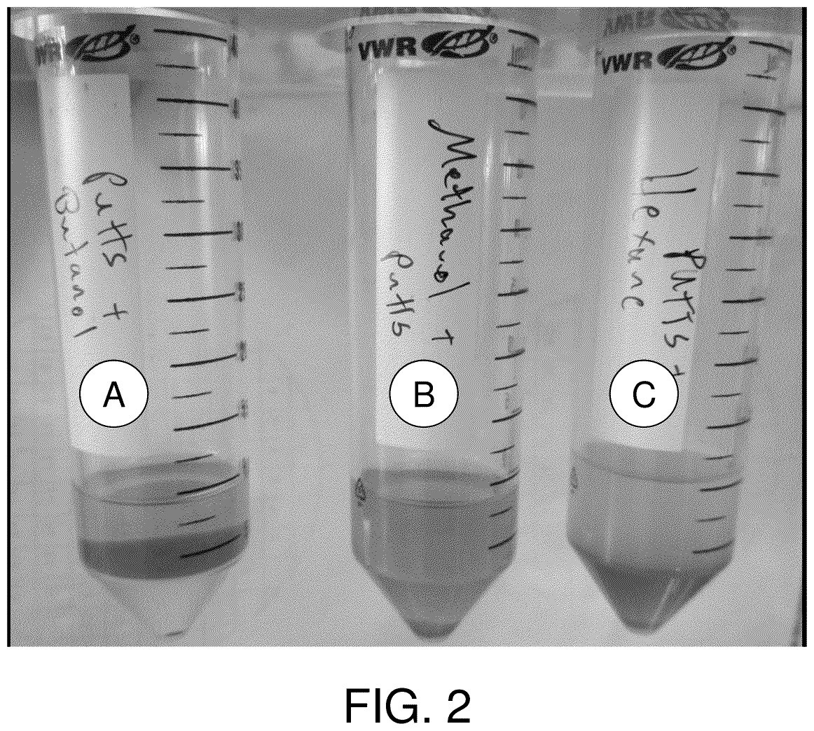

In certain implementations, in a further optional step the alcoholic solvent 24 and supernatant 14 mixture is allowed to settle, is centrifuged, or otherwise seperated (box 110). The mixture settles or is centrifuged (box 110) into three distinct layers a top layer 28, a middle layer 30, and a bottom layer 32. The various layers are shown in FIG. 2

FIG. 2 shows treatment of lyophilized-powder Arthrospira platensis using PUTTS buffer with Butanol (left), Methanol (center), and Hexane (right). As can be seen, treatment with PUTTS and butanol allowed separation of the chlorophyll (upper butanol layer/top layer 28) from the protein fraction (PUTTS fraction/bottom layer 32).

Continuing with FIG. 1, in various implementations, the top layer 32 is an immiscible alcohol layer that visually contains green chlorophyll. The top layer 32 may be aspirated away from the other layers and optionally heated (box 111) to volatilize and recover alcohol solvent 34 and recover chlorophyll powder 36. The heating may be done at various temperatures and times, as would be understood. For example, the top layer 32 may be heated at 114° C. for recovery of butanol. The chlorophyll powder 36 may optionally be purged (box 120) with N₂ gas to prevent oxidative damage.

The purified and dried chlorophyll 36 may optionally be enzymatically processed (box 112) at appropriate conditions to yield tocopherol, pheophorbide B, phytol, or other degradation products. Optionally, the end product may have nutraceutical properties. The enzymes for the enzymatic processing (box 112) may include, but are not limited to, catalase, chlorophyllase, chlorophyll dephytylase, and/or pheophorbide oxygenase (PAO). Various additional or alternative enzymes are possible to yield various end products and would be understood by those of skill in the art.

In a further optional step, the middle layer 30 is aspirated away from the other layers and optionally may undergo acid-catalyzed fatty acid transesterification (box 113) for biodiesel production or other processing of lipoproteins, glycolipids, neutral lipids, or polar or non-polar free fatty acids present. In other implementations, the middle layer 30 can undergo base-catalyzed fatty acid transesterification (box 113). Various additional processing steps for the middle layer 30 are possible and would be recognized by those of skill in the art.

In an additional optional step, the bottom layer 32 may undergo filtration (box 114) to concentrate and purify soluble and insoluble proteins 40 away from denaturants of the solubilization formulation 12. The filtration (box 114) may include crossflow/tangential or dead-end membrane ultra-diafiltration or Centri-prep filtration.

Membranes for use in filtration (box 114) may consist of polymers that include, but are not limited to, polyether sulfonates (PES), polyvinyl difluorides (PVDF), and others with molecular-weight cutoffs that include, but are not limited to, 3 kDa, 10 kDa, and others, as would be appreciated. Further, the transmembrane pressure and crossflow velocity can be adjusted for optimal performance. The filtration conditions and parameters can be adjusted.

As would be understood, the filtration (box 114) may include a single or multiple passes with or without continuous recirculation. After a majority of the feed (bottom layer 32) to the membrane has transferred to the permeate 38 containing recovered solubilization formulation, the permeate 38 is removed (box 115) and recovered to optionally be used as solubilization formulation 12 used in at the start of the process (box 101).

As would be understood, recovered permeate 38 can be recycled for the denaturation process or alternatively in the case of urea, it can be used as a nitrogen source in algal cultivation. Alternative uses are possible and contemplated herein.

In a further optional step, the bottom layer 32 undergoes a secondary diafiltration step (box 116). In this optional step, reverse osmosis or deionized water then added to membrane from the prior filtration step (box 114) to wash, solubilize, and dislodge the proteins 40 that adhere to and foul the membrane and reduce permeate flux and/or remain in the retentate as likely precipitates of filtration (box 116). The final retentate containing mostly protein 40 is obtained and recovered by pooling a final water-wash (box 117) of the membrane with a final water re-suspension of likely precipitates in the feed.

### EXAMPLES

### Example 1

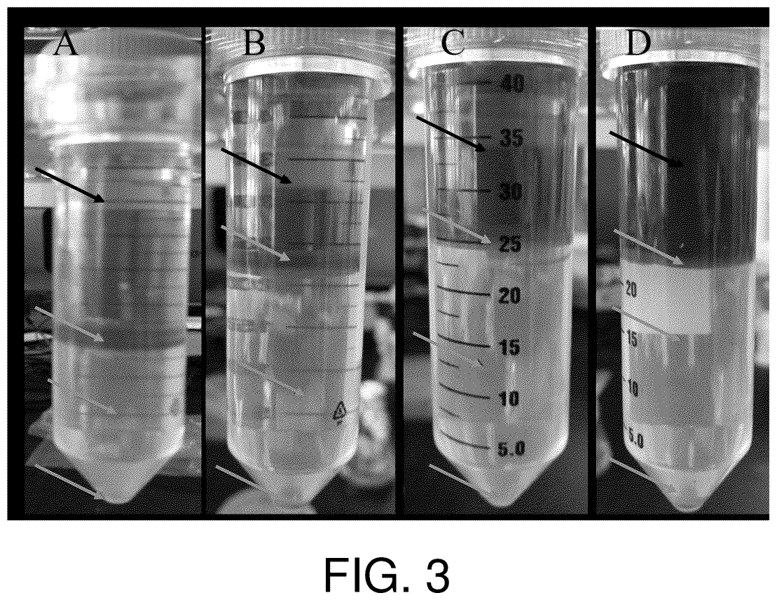

FIG. 3 shows PUTTS:Butanol (1:1) extraction after centrifugation of biomass from A) nitrogen replete Stramenopile algae. B) nitrogen deplete Stramenopile algae, C) nitrogen deplete Muriella zofingeinsis, (previously called Chlorella zofingeinsis), and D) Nitrogen deplete Haematococcus pluvialis. In this example, cells were disrupted with microfluidization and freeze dried prior to extraction of the dried biomass with incubation for 10 min in 23° C. PUTTS buffer followed by addition of butanol and centrifuged at 2500 rpm for 10 min at room temperature. The black arrow denotes butanol layer (top layer) containing chlorophyll (Stramenopile algae) or carotenoid (M zofingeinsis and H pluvialis). The orange arrow indicates lipid rich interphase zone (middle layer). The green arrow indicates the PUTTS layer rich in protein (bottom layer). The blue arrow indicates the decolorized biomass containing cell walls and colorized unbroken cells.

### Example 2

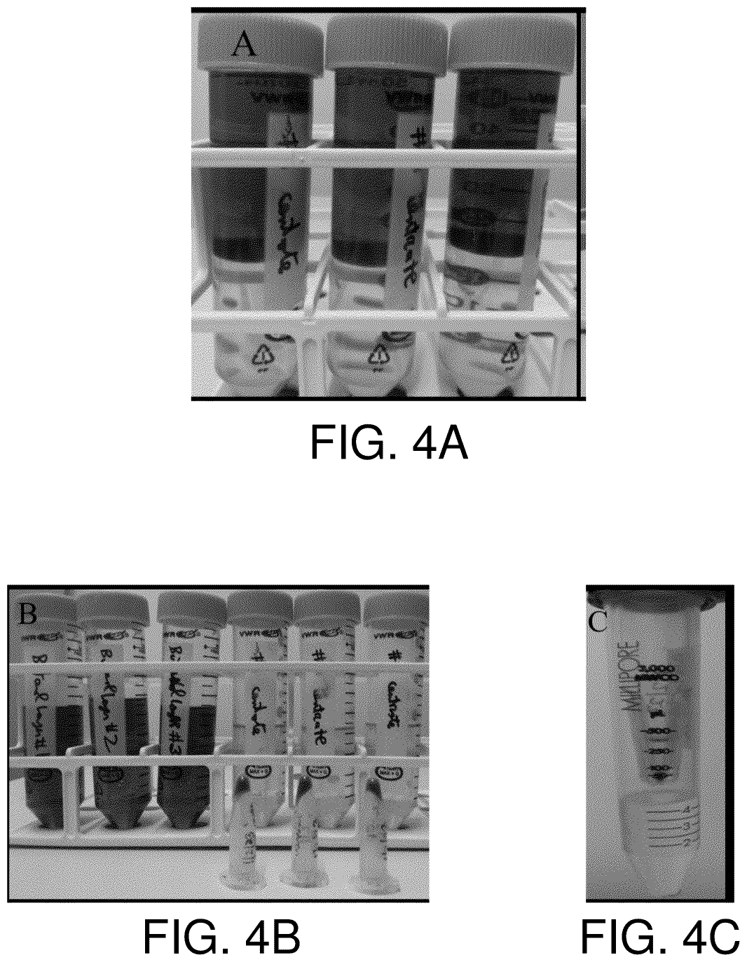

FIG. 4A shows triplicate PUTTS:Butanol (1:1 v:v) extraction of residual raffinate resulting from extraction of water-soluble protein (including phycocyanin) from the biomass of Arthrospria platensis that was PEF-treated, solubilized in PUTTS, centrifuged, and separated as supernatant from a polysaccharide-rich pellet. The triplicate extractions reveal a top green butanol layer containing chlorophyll (top layer), a lipid rich interphase layer (middle layer), and a clear PUTTS layer (bottom layer). FIG. 4B shows separated fractions of butanol (separated top layer) (three tubes on the left); PUTTS layer (separated bottom layer) (central three tubes with clear fractions) also including a washed and hydrolyzed polysaccharide layer; and dried central lipid interphase (1.5 mL Eppendorf tubes in front). FIG. 4C shows the PUTTS layer (bottom layer) after passing through 3000 kD MWCO centrifuge filter. As discussed herein the PUTTS solution passes through the filter while proteins ≥3000 kD remain on the membrane.

### Example 3

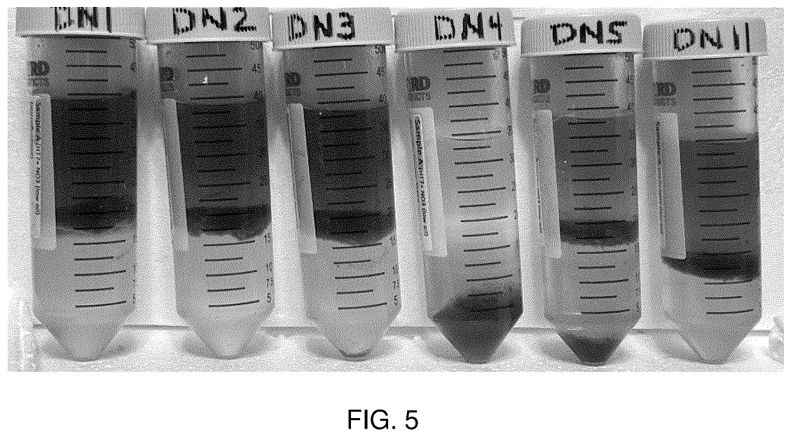

FIGS. 5-23 show extractions of dry microalgal biomasses—Samples A-G—using various (1:1 v:v) combinations of solvent-1 (100% n-butanol):solvent-2 (DN1, DN2, DN3, DN4, DN5, DN6, DN7, DN8, DN9, DN10, and DN11). Sample biomasses and solvent-2 for this example are designated as follows:

TABLE 1
Sample Biomass

| | Biomass |
|---|---|
| Sample A | micro fluidized, N-replete (low-oil) Tribonema viride |
| Sample B | micro fluidized, N-deplete (high-oil), Tribonema viride |
| Sample C | undisrupted, N-replete (low-oil) Tribonema viride |
| Sample D | undisrupted, N-deplete (high-oil), Tribonema viride |
| Sample E | micro fluidized, N-replete Hematococcus pluvialis |
| Sample F | micro fluidized, N-replete Chlorella vulgaris |
| Sample G | micro fluidized, N-replete Porphyridium cruentum |

TABLE 2
Solvent

| | Solvent | Ratio |
|---|---|---|
| DN1 | Choline Acetate (ChoAC) | 1:5 (v:v) |
| | (pH 5.5): H2O) | (volumetric ratio) |
| DN2 | Choline Acetate (pH 5.5): H2O | 1:10 (v:v) |
| | | (volumetric ratio) |
| DN3 | Choline Acetate (pH 5.5): H2O | 1:20 (v:v) |
| | | (volumetric ratio) |
| DN4 | Choline Acetate (pH 5.5): Urea | 1:2 (M:M) |
| | | (molar ratio) |
| DN5 | (Choline Acetate (pH 5.5): Urea | 9:1 (v:v) |
| | at 1:2 (M:M) (molar ratio)): H2O | (volumetric ratio) |
| DN6 | Choline Dihydrogen Phosphate | 1:5 (v:v) |
| | (pH 7.0): H2O | (volumetric ratio) |
| DN7 | Choline Dihydrogen Phosphate | 1:10 (v:v) |
| | (pH 7.0): H2O | (volumetric ratio) |
| DN8 | Choline Dihydrogen Phosphate | 1:20 (v:v) |
| | (pH 7.0): H2O | (volumetric ratio) |
| DN9 | Choline Dihydrogen Phosphate | 1:2 (M:M) |
| | (pH 7.0): Urea | (volumetric ratio) |
| DN10 | (Choline Dihydrogen Phosphate | 9:1 (v:v) |
| | (pH 7.0): Urea at 1:2 (M:M) | (volumetric ratio) |
| | (molar ratio)): H2O | |
| DN11 | PUTTS Buffer | |

### Materials and Methods

Biomasses were obtained via (1) cultivation and harvesting in flat-panel photobioreactors (PBRs) at either N-deplete (no NaNO₃) or N-replete (1.5 g/L NaNO₃) conditions, (2) micro fluidization treatment or no treatment, and (3) final lyophilization-based freeze-drying treatment.

Micro fluidization treatment entailed 200 mL/min flow rate and 25,000 psi at room temperature 23° C., with 3 passes to assure full disruption. An amount of 0.1 g of biomass was weighed and deposited in a 50 ml conical tube, to which 20 mls of the respective solvent-2 was added. Sample tubes were then vortexed vigorously for 5 min and then centrifuged at 4000×g for 30 min at 4° C. Any remaining biomass pellet was photographed to assess solvation efficiency and effectiveness of solvent-2. An amount of 20 mls of solvent-1 (100% butanol) was then added to all solvent-2 extractions. Samples were again vortexed vigorously for 5 min and then centrifuged at 4000×g for 30 min at 4° C. The resulting two-solvent extractions were visualized and photographed. Any remaining biomass pellet at the bottom of the tubes was photographed to assess solvation efficiency and effectiveness of solvent-2.

The 8M urea content in PUTTS Buffer (DN11) was identical to that in DN5 and DN10. Choline acetate (146.2 g) and choline dihydrogen phosphate were formulated with urea (120.2 g) at a 1:2 molar ratio. In both cases of DES (Choline Acetate+Urea and Choline Dihydrogen Phosphate+Urea) used at 1:2 molar ratio for formulations DN4 and DN9, the solvents were very viscous and did not effectively dissolve the biomass. In general, the ionic liquids (ILs) and PUTTS Buffers were effective for both chlorophyll and carotenoid extraction.

### Results

FIG. 5 shows results of extraction for sample A with DN1, DN2, DN3, DN4, DN5, and DN11. As can be seen, for sample A, DN11 resulted in selective extraction of green chlorophyll in the top n-butanol layer that was visually comparable to those of DN1, DN2, and DN3 and visually greater those of the DN4 and DN5. DN4 and DN5 formulations involving ionic liquid/urea (deep eutectic solvent).

For samples A and B, the PUTTS buffer of DN11 denatured and decolored more of the biomass pellet compared to extractions with DN3, DN4, and DN5. For microfluidized samples (samples A, B, E, F, and G) the choline acetate (ChoAC) variations of solvent-2 (DN1-3) performed as well as DN11.

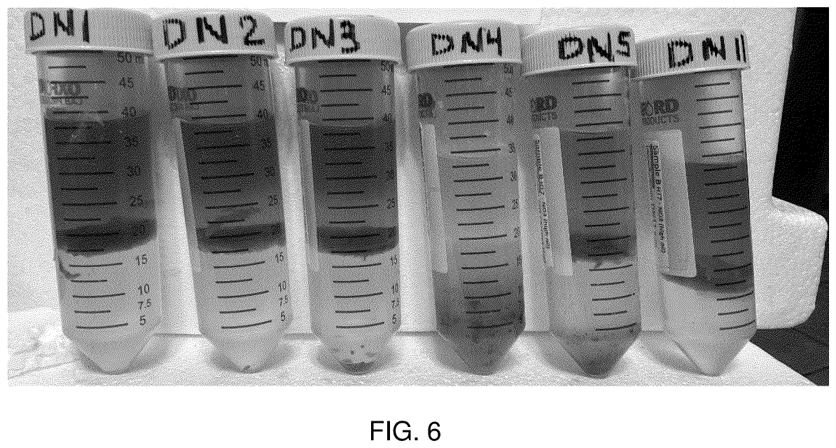

FIG. 6 shows results of extraction for sample B with DN1, DN2, DN3, DN4, DN5, and DN11. Here DN11 resulted in selective extraction of green chlorophyll in the top n-butanol layer that was visually comparable to those of DN1, DN2, and DN3 and visually greater those of the DN4 and DN5.

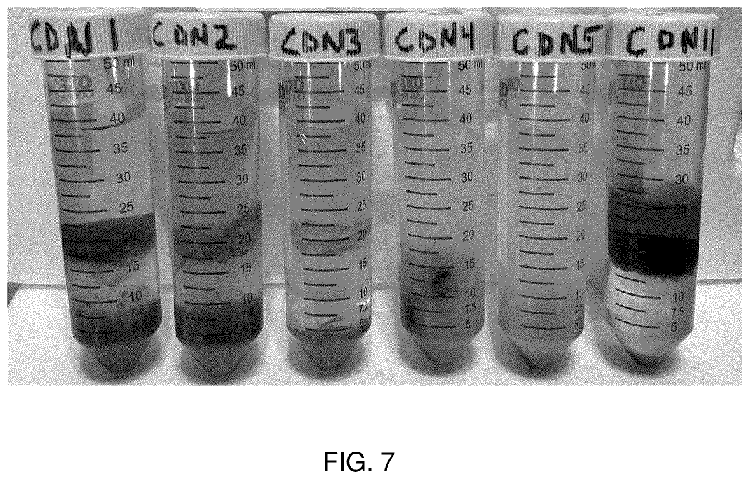

FIG. 7 shows results of extraction for sample C with DN1, DN2, DN3, DN4, DN5, and DN11 Here, DN11 resulted in selective extraction of green chlorophyll in the top n-butanol layer that was visually greater to those of DN1, DN2, and DN3 and visually greater denaturing and decolorization of the biomass pellet compared to DN1, DN2, and DN3. For sample D, extracted chlorophyll in the top layer and middle layer suspected of containing extracted lipids/fatty acids were absent in DN4 and DN5. When undisrupted biomass was used in the denaturant/extraction process, PUTTS Buffer in DN11 performed better than the DES or ILs.

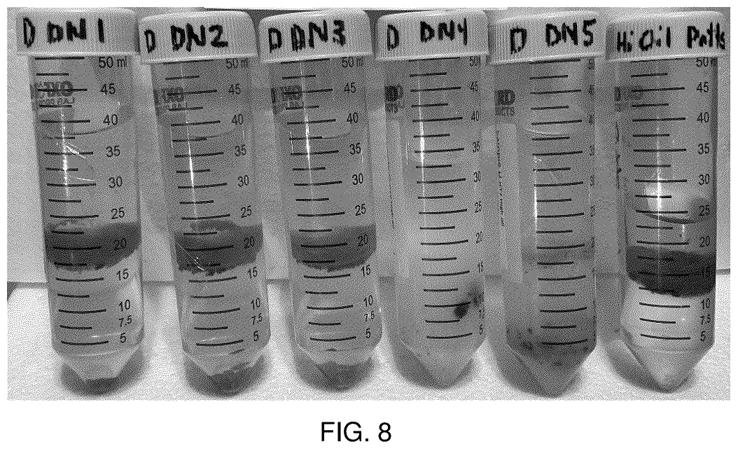

FIG. 8 shows results of extraction for sample D with DN1, DN2, DN3, DN4, DN5, and DN11. Here, DN11 resulted in selective extraction of green chlorophyll in the top n-butanol layer that was visually only slightly greater to those of DN1, DN2, and DN3 and visually greater denaturing and decolorization of the biomass pellet compared to DN1, DN2, and DN3. For sample D, a high-oil biomass, extracted chlorophyll in the top layer and middle layer suspected of containing extracted lipids/fatty acids were absent in DN4 and DN5, despite the absence of a green pellet signifying full denaturing. The results for DN4, DN5 containing DES (both neat and diluted ChoAC) when compared to the PUTTS-buffer-containing DN11 samples, was less effective in extracting chlorophylls and carotenoids from the pellet. However, this is not attributed to the urea, as both had about 8 M urea, but instead to the presence of the surfactants.

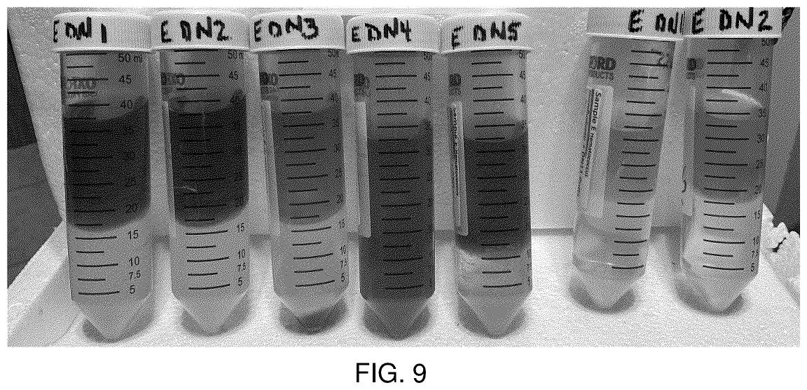

FIG. 9 shows results of extraction for sample E with DN1, DN2, DN3, DN4, DN5, and DN11. Herein, DN11 resulted in selective extraction of green chlorophyll (and, potentially, also brown carotenoid astaxanthin that later oxidized and discolored after being left at room temperature 25° C. for 8 hours) in the top n-butanol layer and decolorization of the biomass pellet that was visually comparable to those of DN1 and DN2. For sample E, DN5 resulted in selective extraction of brown astaxanthin in the top n-butanol layer and decolorization of the pellet in the bottom denaturing layer that was comparable to those of DN1 and DN2 but significantly greater than for DN3. No phase separation was evident for the more viscous DN4 sample.

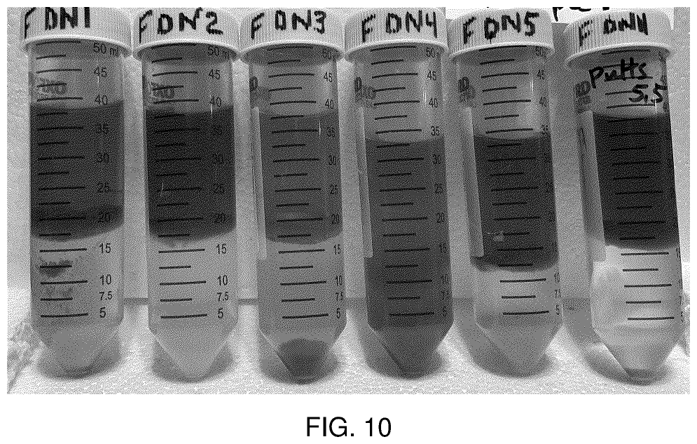

FIG. 10 shows results of extraction for sample F with DN1, DN2, DN3, DN4, DN5, and DN11. Here, DN2 resulted in selective extraction of brown astaxanthin in the top n-butanol layer and decolorization of the biomass pellet that was visually comparable to those of DN1, DN2, DN5, DN11. For sample F, DN2 resulted in the greatest denaturing and decolorization of bottom layer biomass pellet compared to urea-containing DN11 and significantly greater than the more concentrated DN3. No phase separation was apparent for DN4. For sample F, the middle layer was less apparent than for samples A, B, and C for all 2-solvent extractions.

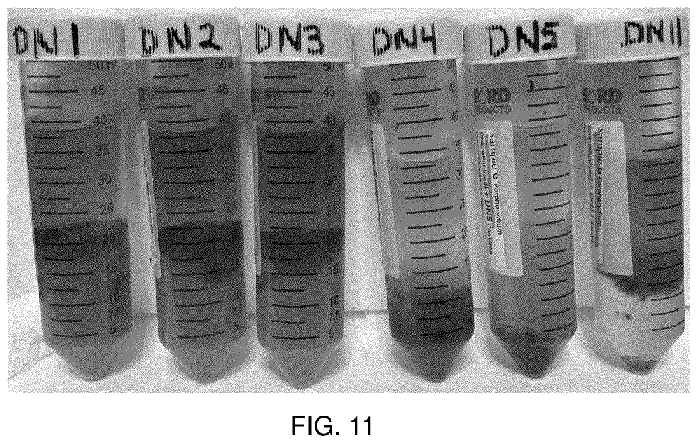

FIG. 11 shows results of extraction for sample G with DN1, DN2, DN3, DN4, DN5, and DN11. Here, DN11 resulted in extraction of visually comparable amounts of green chlorophyll in the top n-butanol layer compared to DN1, DN2, and DN3, but less denaturing and decolorization of bottom layer biomass pellet compared to DN1, DN2, and DN3. Furthermore, DN1, DN2, and DN3 resulted in extraction and preservation of the three-dimensional conformation of phycoerythrin water-soluble phycobiliprotein, as evidenced by the pink coloration in the bottom layer. In contrast, DN11, DN4, and DN5 bottom layers are devoid of any red or pink coloration, suggesting that the urea denatured the phycoerythrin. The middle layer of DN11 was also smaller than those of DN1, DN2, and DN3. No chlorophyll or phase separation visibly occurred in DN5-mediated extraction. In these Porphyridium cruentum biomass trials, ChoAC (diluted) facilitated further removal of chlorophyll while maintaining the red color in the bottom layer solvent.

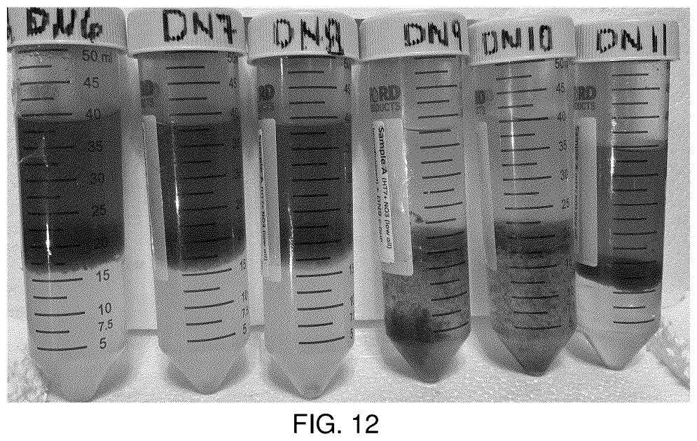

FIG. 12 shows results of extraction for sample A with DN6, DN7, DN8, DN9, DN10, and DN11. Here, DN11 resulted in visually comparable green chlorophyll extraction in the top n-butanol layer and decolorization and denaturing of the bottom layer pellet compared to DN6, DN7, and DN8. For sample A, DN9 and DN10, which both contained C-DHP and urea, exhibited no chlorophyll extraction and the least denaturing of the biomass pellet, as evidenced by substantial green pellets still present. Middle layers were comparable in size for all samples.

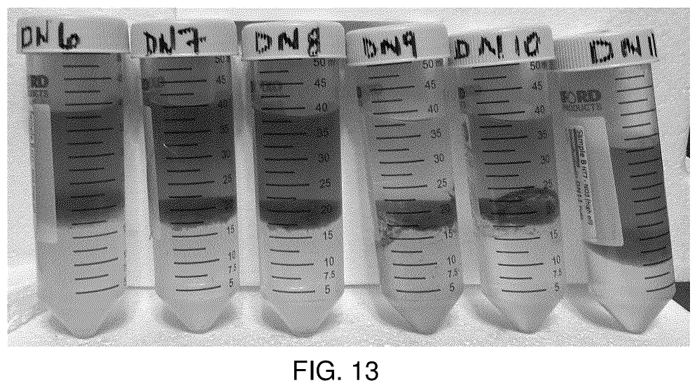

FIG. 13 shows results of extraction for sample B DN6, DN7, DN8, DN9, DN10, and DN11. Here, DN11 resulted in low but visually comparable green chlorophyll extraction in the top n-butanol layer and decolorization and denaturing of the bottom layer pellet compared to DN6, DN7, and DN8. For sample B, DN9 and DN10 exhibited no chlorophyll extraction Middle layers were comparable in size for all samples.

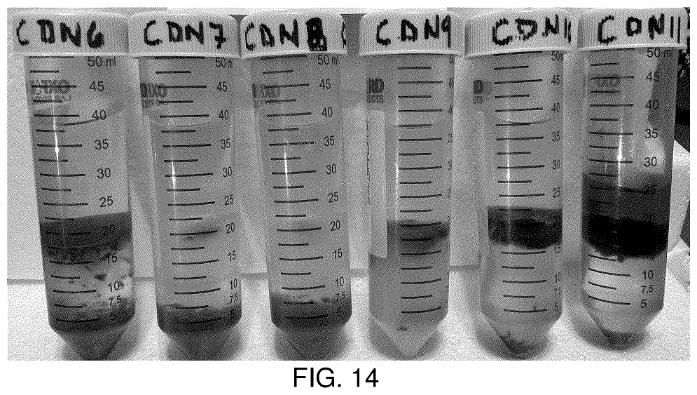

FIG. 14 shows results of extraction for sample C with DN6, DN7, DN8, DN9, DN10, and DN11. Here, DN11 and DN10, both containing urea, resulted in the greatest and only visibly apparent chlorophyll extraction, as well as the most substantial middle layer and the most denaturing and decolorization of biomass bottom pellet compared to DN6, DN7, DN8, and DN9.

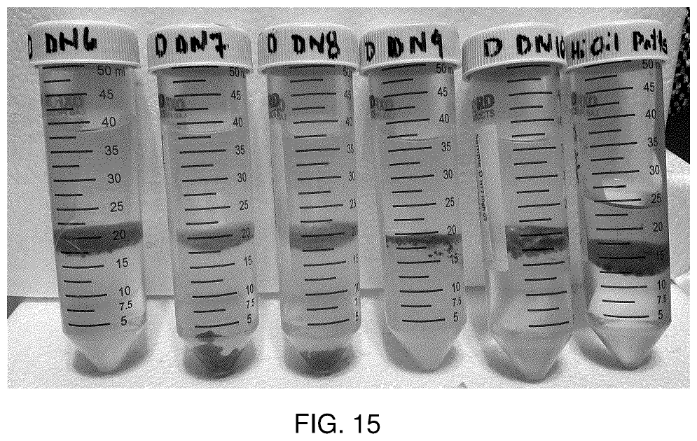

FIG. 15 shows results of extraction for sample D with DN6, DN7, DN8, DN9, DN10, and DN11 Here, DN11, containing urea, resulted in the greatest and only visibly apparent chlorophyll extraction, as well as the most substantial middle layer compared to DN6, DN7, DN8, DN9, and DN10. Only DN7 and DN8 revealed an intact green bottom layer biomass pellet, while pellets were absent in DN6, DN9, DN10, and DN11. The extractions with DN9, DN10 containing DES (both neat and diluted ChoDP) when compared to the PUTTS-buffer-containing DN11 samples, appear to be less effective in extracting chlorophylls and carotenoids from the pellet. However, this is not attributed to the urea, as both had about 8 M urea, but instead to the presence of the surfactants.

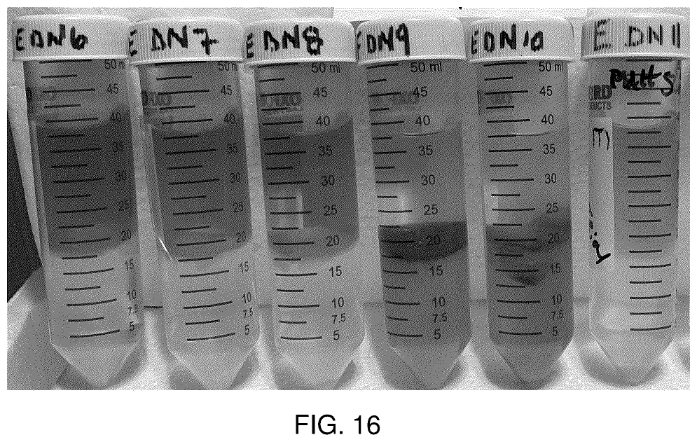

FIG. 16 shows results of extraction for sample E with DN6, DN7, DN8, DN9, DN10, and DN11 Here, DN11 resulted in selective extraction of green chlorophyll (and, potentially, also brown carotenoid astaxanthin that later oxidized and discolored) in the top n-butanol layer and decolorization of the biomass pellet that was visually comparable to those of DN6, DN7, and DN8. For sample E, DN9 and DN10 resulted in phase separation with an evident middle layer. However, the carotenoid astaxanthin was uniformly distributed in both bottom and lower layer in DN9 and DN10.

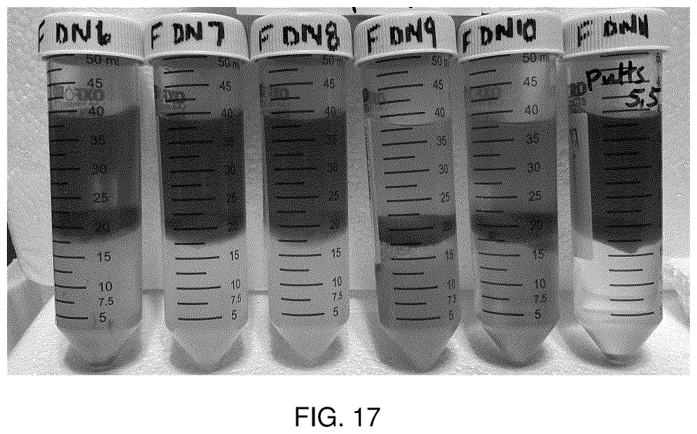

FIG. 17 shows results of extraction for sample F with DN6, DN7, DN8, DN9, DN10, and DN11 Here, DN11 resulted in selective extraction of brown astaxanthin in the top n-butanol layer and decolorization of the biomass pellet that was visually comparable to those of DN6, DN7, DN8. Decolorization of bottom layer pellet was less in DN11 and DN6 compared to those of DN7, DN8, DN9, and DN10. For sample F, DN9 and DN10 resulted in phase separation with an evident middle layer. However, the carotenoid astaxanthin was uniformly distributed in both bottom and lower layer in DN9 and DN10.

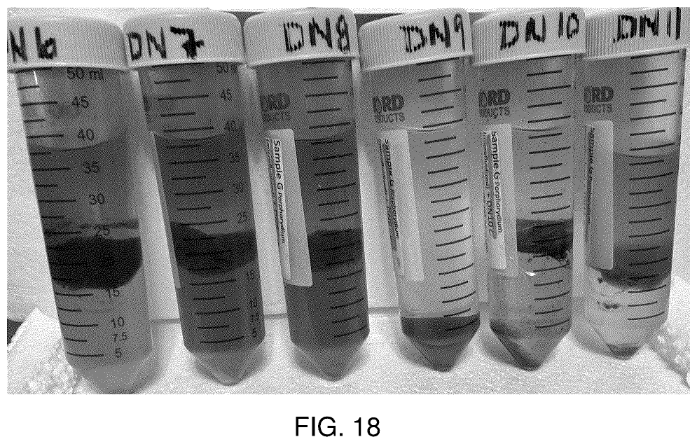

FIG. 18 shows results of extraction for sample G with DN6, DN7, DN8, DN9, DN10, and DN11. Here, a visibly white, decolorized pellet and red bottom layer for extractions of sample G involving DN1, DN2, DN3, DN6, DN7, DN8, and DN10 signify the preservation of the three-dimensional structure of the red phycobiliprotein yet extraction of colorants (i.e. phycobiliproteins and chlorophylls) from the biomass (extractions with DN1, DN2, and DN3 are shown in FIG. 11). The maintenance of the red/pink/purple color in the bottom layer of the extracts of Porphyridium is indicative of the protein staying in the lower layer and not migrating to the butanol layer.

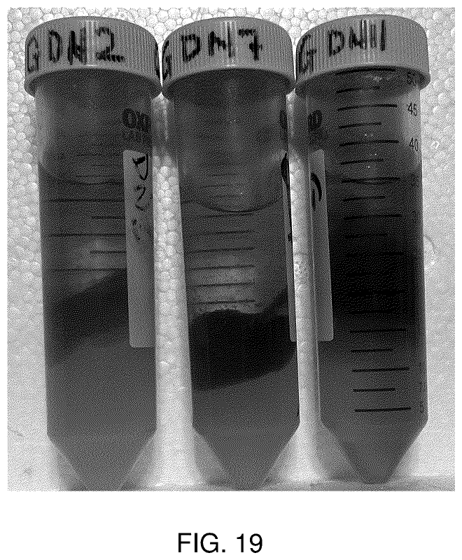

FIG. 19 shows results of extraction for sample G with DN2, DN7, and DN11. In these extractions, denaturing of the red phycobiliprotein (phycoerythrin) by urea and/or surfactants is evidenced by the purple coloration of the bottom layers of the DN7 and DN11 extractions. In contrast, the hot-pink color of the bottom layer of the DN2 extraction signifies the preservation of the three-dimensional structure of the red phycobiliprotein yet thorough extraction of all colorants (i.e. phycobiliproteins and chlorophylls) from the biomass. Further in the DN2 and DN11 extractions, the green color of the top n-butanol layer signified thorough and greater extraction of intact green chlorophyll compared to the DN7 extraction.

What is claimed is:

1. A chemical extraction method comprising:
exposing a biomass to a protein-denaturing solution to produce a suspended biomass mixture;
separating the solubilized biomass mixture into a supernatant phase and a decolorized biomass phase;
hydrolyzing the decolorized biomass phase into a hydrolyzed product;
fermenting the hydrolyzed product into an alcoholic solvent;
mixing the alcoholic solvent and supernatant phase to produce a product suspension; and
fractioning the product suspension into a bottom phase, middle phase, and top phase.

2. The chemical extraction method of claim 1, wherein separating the solubilized biomass mixture is accomplished with gravimetric settling.

3. The chemical extraction method of claim 1, wherein separating the solubilized biomass mixture is accomplished with centrifugation.

4. The chemical extraction method of claim 1, further comprising:
washing the solubilized biomass mixture; and
re-exposing the solubilized biomass mixture to a protein-denaturing solution.

5. The chemical extraction method of claim 1, wherein the alcoholic solvent is butanol.

6. The chemical extraction method of claim 1, wherein fractioning the product suspension is accomplished by gravimetric settling.

7. The chemical extraction method of claim 1, further comprising:
filtering the bottom layer into proteins and permeate;
transesterifying the middle phase with fatty esters; and
drying the top layer into a powder.

8. The chemical extraction method of claim 7, wherein the powder is substantially chlorophyll.

9. The chemical extraction method of claim 8, further comprising enzymatically processing the powder into a tocopherols, pheophorbide B, or phytol.

10. The chemical extraction method of claim 1, wherein the protein-denaturing solution is a PUTTS buffer.

11. The chemical extraction method of claim 1, wherein the biomass is one or more of macroalgae, microalgae, cyanobacteria, and terrestrial plants.

12. A biomass separation system comprising:
(a) an agitation device configured to contact a biomass and a PUTTS solution to produce a solubilized biomass mixture;
(b) a first separation device capable of separating the solubilized biomass mixture based on density into a supernatant and decolorized biomass;
(c) a hydrolysis reactor configured to hydrolyze the decolorized biomass;
(d) a fermenter configured to ferment the decolorized biomass into an alcoholic solution;
(e) a mixing vessel configured mix the alcoholic solution and supernatant into a product suspension; and
(f) a second separation device for separating the product suspension into a bottom phase, middle phase, and top phase.

13. The biomass separation system of claim 12, wherein the first separation device is a centrifuge or a settling tank.

14. The biomass separation system of claim 12, further comprising a filter configured to filter the bottom layer into proteins and permeate.

15. The biomass separation system of claim 12, further comprising a dryer configured to separate the top layer into alcohol solvent and a powder.

16. The biomass separation system of claim 12, wherein the powder is substantially chlorophyll.

17. The biomass separation system of claim 12, further comprising an enzymatic reactor configured to enzymatically react the chlorophyll into tocopherols, pheophorbide B, or phytol.

18. A method for fractioning photosynthetic biomass, comprising:
solubilizing photosynthetic biomass in a first solvent;
separating the solubilized photosynthetic biomass into a supernatant and decolorized biomass;
hydrolyzing the decolorized biomass;
fermenting the hydrolyzed decolorized biomass;
missing the second solvent with the supernatant;
fractioning the supernatant mixed with the second solvent into top, middle, and bottom layers; and
extracting chlorophyll, catenoids, lipids and fatty acids, proteins, polysaccharides from the top, middle, and bottom layers,
wherein the first solvent is selected from a relatively hydrophilic aqueous surfactant—and protein denaturant-based solvent, a relatively hydrophilic aqueous ionic liquid-based solvent, a relatively hydrophilic aqueous surfactant—and/or protein denaturant-based deep eutectic solvent, and an immiscible, relatively hydrophobic deep eutectic solvent,
wherein the second solvent is selected from an immiscible, relatively hydrophobic polar alcohol;
an immiscible, relatively hydrophobic polymer-based solvent; an immiscible, relatively hydrophobic aqueous ionic liquid-based solvent; an immiscible, relatively hydrophobic, deep eutectic solvent; and an immiscible, relatively hydrophobic aqueous ionic liquid-based solvent.

19. The method of claim 18, wherein the first solvent is PUTTS buffer and the second solvent is n-butanol.

20. The method of claim 19, wherein deep eutectic solvents are used to de-water and recover polar alcohol solvents.
